(19) United States
(12) Patent Application Publication
(10) Pub. No.: US 2020/0000705 A1
(43) Pub. Date: Jan. 2, 2020

(54) Aloe smooth skin after shave baume-bump less

(71) Applicant: Melwyn Gaius Turner, Racine, WI (US)

(72) Inventor: Melwyn Gaius Turner, Racine, WI (US)

(21) Appl. No.: 16/501,190

(22) Filed: Mar. 5, 2019

Related U.S. Application Data
(60) Provisional application No. 62/710,752, filed on Apr. 20, 2018.

(51) Int. Cl. A61K 8/9794; A61K 8/34; A61K 8/86; A61K 8/9789; A61Q 9/02

(52) U.S. Cl. CPC A61K 8/9794; A61K 8/34; A61Q 9/02; A61K 8/9789; A61K 8/86

(57) ABSTRACT

A composition for the elimination of Razor Bumps and Skin Irritations where in the combination of Aloe Vera Extract, Melaleuca Alternifolia and Polyethylene Glycol are combined to treat and eliminate Razor Bumps and Skin Irritations.

### BACKGROUND OF THE INVENTION

The composition's design was designed to suppress the appearance of Shaving Bumps and skin blemished irritations. This specially designed Baume soothes, comforts and hydrates the skin. It creates a skin which looks and feel healthier. Overall it's a great product for sensitive and reactive skin which is prone to Bump after shaving. The invention of this product will help to protect the skin against “silent” skin conditions.

Baume de Visage By Mel® was developed due to the dissatisfaction with the products that were being used personally to prevent skin irritations and After Shave Bumps. After many years of using products over the counter it was decided to launch out and formulate a product which would prevent razor Shaving Bumps and leave the skin absent of irritations and after shaving Bumps. After many trials of formulating with a variety of many chemicals and testing a product was developed that would keep the skin supple, smooth free of Bumps and a healthy look and feel. Finally, the product was developed to perfection after more than fifteen years (15) of personal use and trials with other users. It's analgesic and natural healing properties aid in moisture retention and serves as a natural astringent.

The product also provides relief from minor burns, relief from insect bites, helps sooth skin irritations, itching and skin abrasions. The unique design of the product protects from ingrown hairs due to shaving. It is an excellent After Shave Conditioner and aid in drying up blemishes on the skin.

The composition enhances protection of the skin against unwanted blemishes as shaving bumps and skin irritations. The embodying of several chemicals components produces an eminent invention:

Aloe Extract, Melaleuca Alternifolia, Polyethylene Glycol, Distilled Water, Ethanol and Fragrance

The composition is processed in two (2) stages. The first stage consists of blending:

0.2%-40% By weight Ethanol

0.2% 40% By weight Polyethylene Glycol

0.2%-40% By weight Melaleuca Alternifolia

0.2%-40% By weight Distilled Water

0.2%-40% By weight Fragrance

The first stage is blended into the second stage with agitation until a homogeneous state is achieved.

The second stage consists of:

0.2%-40% By weight Aloe Vera Extract

The claimed invention is:

1. An After Shave Baume composition for the elimination of “After Shave Bumps” and skin irritations.
0.2%-40% By weight Ethanol
0.2%-40% By weight Polyethylene Glycol
0.2%-40% By weight Melaleuca Alternifocia
0.2%-40% By weight Aloe Vera Extract
0.2%-40% By weight Distilled Water

2. The composition as defined in claim one (1) further includes a fragrance

3. To minimize Razor Bumps and Skin Irritations apply the composition to the shaves area as you would an after shave lotion.
